(19) United States
(12) Patent Application Publication
(10) Pub. No.: US 2026/0232205 A1
(43) Pub. Date: Aug. 13, 2026

(54) PHYSIOLOGICAL SIGNAL DETECTION AND CORRELATION

(71) Applicant: iRhythm Technologies, Inc., San Francisco, CA (US)

(72) Inventors: Ryan Wensley, San Rafael, CA (US); Steven Robert Keyes, San Francisco, CA (US)

(73) Assignee: iRhythm Technologies, Inc., San Francisco, CA (US)

(21) Appl. No.: 19/462,691

(22) Filed: Jan. 28, 2026

Related U.S. Application Data
(60) Provisional application No. 63/755,944, filed on Feb. 7, 2025.

(51) Int. Cl. A61B 5/0205; A61B 5/00; A61B 5/024; A61B 5/1455; A61B 5/352; A61B 5/355

(52) U.S. Cl. CPC A61B 5/0205; A61B 5/14552; A61B 5/352; A61B 5/355; A61B 5/6833; A61B 5/7246; A61B 5/02438; A61B 2560/0223

(57) ABSTRACT

Techniques for physiological signal detection and correlation are described. In one or more implementations, a first physiological signal corresponding to a first physiological measurement and a second physiological signal corresponding to a second physiological measurement are collected. A first feature of the first physiological signal is detected. Based on an expected temporal correspondence between the first feature and the second physiological signal, a second feature of the second physiological signal is detected. In an example, the first physiological signal is an electrocardiogram (ECG) waveform corresponding to an ECG measurement, and the second physiological signal is a photoplethysmogram (PPG) waveform corresponding to a PPG measurement.

### RELATED APPLICATIONS

This application claims priority to U.S. Provisional Application No. 63/755,944, filed Feb. 7, 2025, and titled “Physiological Signal Detection and Correlation,” which is hereby incorporated by reference in its entirety.

### BACKGROUND

Physiological monitoring devices often utilize multiple sensors to measure various biological signals of users, such as electrocardiogram (ECG) and photoplethysmogram (PPG) waveforms. These signals contain relevant information about cardiovascular health and other physiological parameters. However, accurately detecting and analyzing specific features within individual signal waveforms can be challenging, particularly when signal quality is poor or artifacts are present. Existing methods for processing these signals typically analyze each waveform independently, which limits an ability to extract reliable information in suboptimal measurement conditions.

### BRIEF DESCRIPTION OF THE DRAWINGS

FIG. 1 is a block diagram of a non-limiting example of an environment that is operable to employ physiological signal detection and correlation techniques as described herein.

FIG. 2 depicts a non-limiting example of a monitoring device.

FIG. 3 depicts a first waveform diagram illustrating a temporal relationship between electrocardiogram (ECG) and photoplethysmogram (PPG) signals, such as recorded by the monitoring device.

FIG. 4 depicts a second waveform diagram illustrating a temporal relationship between electrocardiogram (ECG) and photoplethysmogram (PPG) signals.

FIG. 5 depicts a diagram illustrating the detection of time offsets between ECG and PPG signals.

FIG. 6 illustrates a method for detecting features in physiological signals using cross-signal temporal correlations.

FIG. 7 illustrates a method for calibrating a timing offset between physiological signals.

### DETAILED DESCRIPTION

Conventional physiological monitoring systems often analyze individual signal waveforms independently, which can limit an ability to extract reliable information, such as when signal quality is poor. For example, photoplethysmogram (PPG) signals used to calculate blood oxygen saturation (SpO2) often have low signal-to-noise ratios, which makes it difficult to accurately detect peaks (e.g., local maxima) and/or troughs (e.g., local minima) of the PPG signal, which are used to calculate SpO2. While increasing LED current can improve signal quality, this approach is power-intensive and may not be feasible for wearable devices with limited battery capacity.

Accordingly, the techniques described herein leverage correlations between different physiological signals to enhance feature detection and measurement accuracy. In an example, a first physiological signal, such as an electrocardiogram (ECG) waveform, is collected along with a second physiological signal, such as a PPG waveform. One or more features are identified in the first physiological signal, such as R-wave peaks and/or T-wave peaks in the ECG waveform. These features, for instance, are used to detect corresponding features in the second physiological signal based on expected temporal relationships. For example, a timing of an ECG R-wave peak can be used as a reference point to locate a corresponding peak in a PPG waveform. Similarly, a timing of a T-wave peak can be used as a reference point to locate a corresponding trough in the PPG waveform. In some examples, the techniques described herein are further usable to calibrate a timing offset between the first physiological signal and the second physiological signal for data collection and/or analysis.

This approach supports robust detection of PPG peaks and troughs in conditions where a low signal-to-noise ratio is common, such as due to motion artifacts, poor sensor contact with skin, low perfusion states, skin pigmentation variations, and/or ambient light interference. By way of example, ECG signals are less susceptible to these conditions than PPG signals, and so using ECG timing as a reference may narrow the search window for PPG features and reduce false peak and trough detections caused by noise. The enhanced feature detection of these techniques thus supports accurate calculation of physiological parameters that are based on PPG values, such as SpO2. For instance, a blood oxygen saturation value may be calculated based on the peaks and troughs identified using the ECG timing as a reference. The correlated signal analysis also provides redundancy, supports quality assessment of the measurements, and conserves power for wearable devices that implement these techniques by avoiding increased LED current and/or additional light sources.

In one or more implementations, an analysis platform performs real-time or near real-time processing of the physiological data, as the data is received from the wearable device. As used herein, the term “real-time” may refer to the processing or analysis of data as the data is received and/or generated, without intentional delay between data collection and processing. The term “near real-time” may refer to the processing or analysis of data with minimal delay (e.g., milliseconds, seconds, or minutes) between data collection and processing. Real-time or near real-time processing, for instance, may enable an immediate or near-immediate response to a detected feature, such as detecting PPG features within seconds or sub-seconds of detecting ECG timing information. Real-time or near real-time processing may include processing data in a streaming fashion as the data arrives, rather than waiting for data collection to be completed before beginning analysis. By way of example, the analysis platform may analyze incoming physiological data streams in real-time or near real-time during an ongoing observation period to detect ECG features and use the identified ECG features to locate corresponding PPG features. This real-time or near real-time processing enables the analysis platform to continuously or semi-continuously (e.g., at a predetermined frequency) correlate physiological signals and calculate physiological parameters such as blood oxygen saturation.

The techniques described herein improve how the wearable device and/or the analysis platform process physiological signals. By way of example, using ECG timing information to locate PPG features enables accurate feature detection even when PPG signal quality is poor. This represents a change to the operation of the wearable device that improves its technical functioning by enabling accurate physiological measurements in low signal-to-noise ratio conditions without increasing LED current or relying on additional light sources, for example. The physiological parameters calculated through this process provide a technical improvement over conventional physiological monitoring systems that analyze PPG waveforms independently, enabling more accurate SpO2 calculations and more efficient power utilization. Accordingly, the techniques described herein improve the functioning of a physiological monitoring system, such as a wearable device with finite battery power.

In this way, the multi-signal approach described herein overcomes limitations of conventional techniques that are reliant on single-waveform analysis to support reliable physiological monitoring in varying real-world conditions. These techniques provide a specific technical improvement to physiological monitoring systems by using temporal correlations between distinct sensor modalities to enhance signal processing accuracy, resulting in more reliable physiological measurements from wearable devices.

In some aspects, the techniques described herein relate to a method for temporal correlation of physiological signals, including: collecting a first physiological signal that corresponds to a first physiological measurement and a second physiological signal that corresponds to a second physiological measurement; detecting a first feature of the first physiological signal; and detecting a second feature of the second physiological signal based on an expected temporal correspondence between the first feature and the second physiological signal.

In some aspects, the techniques described herein relate to a method, wherein the first physiological signal is collected from a first sensor of a wearable device, and the second physiological signal is collected by a second sensor of the wearable device or a separate wearable device.

In some aspects, the techniques described herein relate to a method, wherein the first physiological signal is an electrocardiogram (ECG) waveform corresponding to an ECG measurement, and the second physiological signal is a photoplethysmogram (PPG) waveform corresponding to a PPG measurement.

In some aspects, the techniques described herein relate to a method, wherein the PPG waveform includes one or more of an infrared (IR) wavelength channel or a red wavelength channel.

In some aspects, the techniques described herein relate to a method, wherein the first feature is an ECG feature of the ECG waveform, and the second feature is a PPG feature of the PPG waveform.

In some aspects, the techniques described herein relate to a method, wherein the ECG feature includes an R-wave peak, and the PPG feature includes a systolic peak.

In some aspects, the techniques described herein relate to a method, wherein the ECG feature includes a T-wave peak, and the PPG feature includes a diastolic trough.

In some aspects, the techniques described herein relate to a method, wherein the first feature includes one or more of a peak, a trough, a complex, or an interval of the ECG waveform, and the second feature includes one or more of a PPG peak or a PPG trough of the PPG waveform.

In some aspects, the techniques described herein relate to a method, further including: calculating a blood oxygen saturation value based at least on the second feature.

In some aspects, the techniques described herein relate to a method, further including calibrating a timing offset between the first physiological signal and the second physiological signal.

In some aspects, the techniques described herein relate to a method, wherein calibrating the timing offset includes: detecting a high signal-to-noise ratio segment of the second physiological signal; determining the timing offset between the first physiological signal and the second physiological signal based on the high signal-to-noise ratio segment; and detecting the second feature further based on the timing offset.

In some aspects, the techniques described herein relate to a system for temporal correlation of physiological signals, including: a first sensor configured to collect a first physiological signal corresponding to a first physiological measurement; a second sensor configured to collect a second physiological signal corresponding to a second physiological measurement; and a processor configured to execute a signal feature correlation algorithm stored in a non-transitory computer-readable storage medium to perform operations including: detecting a first feature of the first physiological signal, and detecting a second feature of the second physiological signal based on an expected temporal correspondence between the first feature and the second physiological measurement.

In some aspects, the techniques described herein relate to a system, wherein the first sensor includes one or more electrodes configured to collect an electrocardiogram (ECG) signal corresponding to an ECG measurement, and the second sensor includes an optical sensor configured to collect a photoplethysmogram (PPG) signal corresponding to a PPG measurement.

In some aspects, the techniques described herein relate to a system, wherein the optical sensor includes: a first light source configured to emit light at a red wavelength; a second light source configured to emit light at an infrared wavelength; and at least one photodetector configured to detect light reflected from tissue.

In some aspects, the techniques described herein relate to a system, wherein the operations further include: calculating a blood oxygen saturation value based at least on the second feature.

In some aspects, the techniques described herein relate to a system, wherein the operations further include: calibrating a timing offset between the first physiological signal and the second physiological signal based on a high signal-to-noise ratio segment of the second physiological signal.

In some aspects, the techniques described herein relate to a system, wherein detecting the second feature includes defining a search window based on a timing of the first feature and locating a local maximum or a local minimum within the search window.

In some aspects, the techniques described herein relate to a processing device, including: one or more processors; and a memory having stored computer-readable instructions that are executable by the one or more processors to perform operations including: receiving a first physiological signal corresponding to a first physiological measurement and a second physiological signal corresponding to a second physiological measurement; detecting a first feature of the first physiological signal; and detecting a second feature of the second physiological signal based on an expected temporal correspondence between the first feature and the second physiological measurement.

In some aspects, the techniques described herein relate to a processing device, wherein the first physiological signal is an electrocardiogram (ECG) waveform corresponding to an ECG measurement and the second physiological signal is a photoplethysmogram (PPG) waveform corresponding to a PPG measurement, and wherein the operations further include: calculating a blood oxygen saturation value based at least on the second feature.

In some aspects, the techniques described herein relate to a processing device, wherein detecting the second feature includes defining a search window based on a timing of the first feature and a calibrated timing offset, and locating a local maximum or a local minimum within the search window.

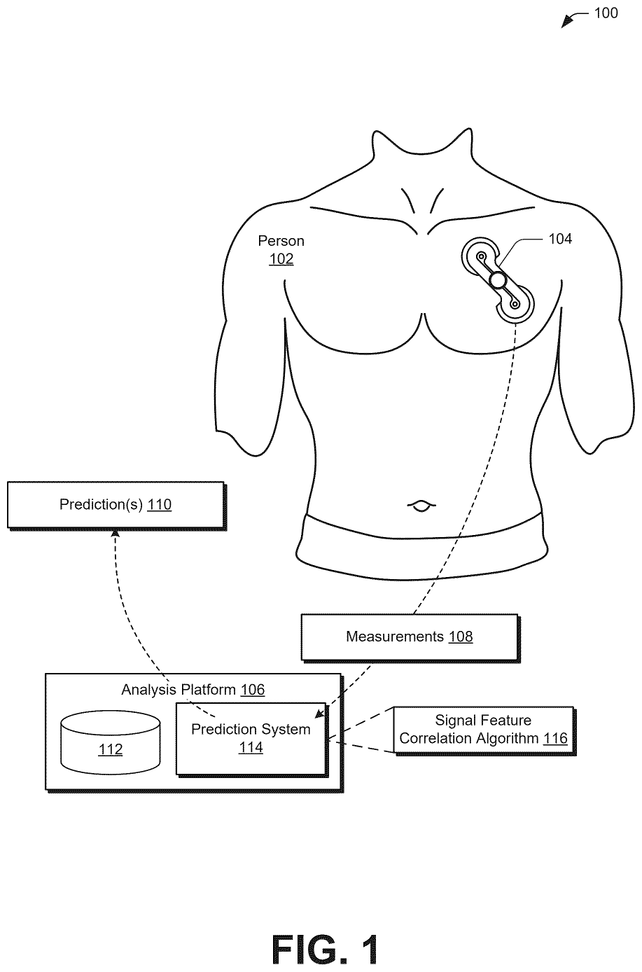

FIG. 1 is a block diagram of a non-limiting example 100 of an environment that is operable to employ physiological signal detection and correlation techniques as described herein. The illustrated example 100 includes a monitored subject, e.g., person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. By way of example, the monitoring device 104 may be utilized to monitor one or more of electrocardiography (ECG), electroencephalography (EEG), electromyography (EMG), respiratory inductance plethysmography (RIP), pulse oximetry, accelerometry, impedance cardiography (ICG), or the like as measurements 108. The monitoring device 104 of the illustrated example 100 includes a PPG sensor for non-invasive monitoring of various physiological parameters. By way of example, the PPG sensor may be utilized to monitor one or more of pulse rate, heart rate variability, blood oxygen saturation, respiration, blood volume, blood perfusion, and blood pressure. The PPG sensor may comprise one or more light sources, such as light-emitting diodes (LEDs) and/or laser diodes, and one or more photodetectors (e.g., a photodiode), as will be elaborated herein. In one or more implementations, the PPG sensor is configured to emit light at and detect multiple different wavelengths of light, such as both red and IR light.

In one or more implementations, the monitoring device 104 may combine PPG sensing with other modalities, such as ECG and/or ICG, to provide a more comprehensive picture of the physiological state of the person 102. This multi-modal approach may enhance the ability of the monitoring device 104 to detect and monitor various health conditions, including sleep disorders, arrhythmias, hypertension, and/or changes in cardiovascular function.

In some scenarios, for instance, the monitoring device 104 may be provided to record electrical activity of the heart of the person 102 over an observation period, e.g., lasting some number of seconds or minutes, lasting multiple days, and so on. By way of example, the electrical activity of the heart of the person 102 may be monitored over time to produce one or more electrocardiograms, which may be used to predict any of a variety of events. Alternatively, or in addition, the monitoring device 104 may be used to output the measurements 108 (e.g., a time sequence of measurements such as a time sequence of electric potential measurements), which may indicate an observation or be used to generate an assessment, diagnosis, or prediction of one or more events. In connection with the monitoring device, instructions may be provided to the person 102 that instruct the person 102 how to operate the monitoring device 104 and/or how to behave (e.g., sleep, perform activity) while wearing the monitoring device 104. In one or more implementations, the instructions may be provided as part of a kit, e.g., written instructions. Alternatively, or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

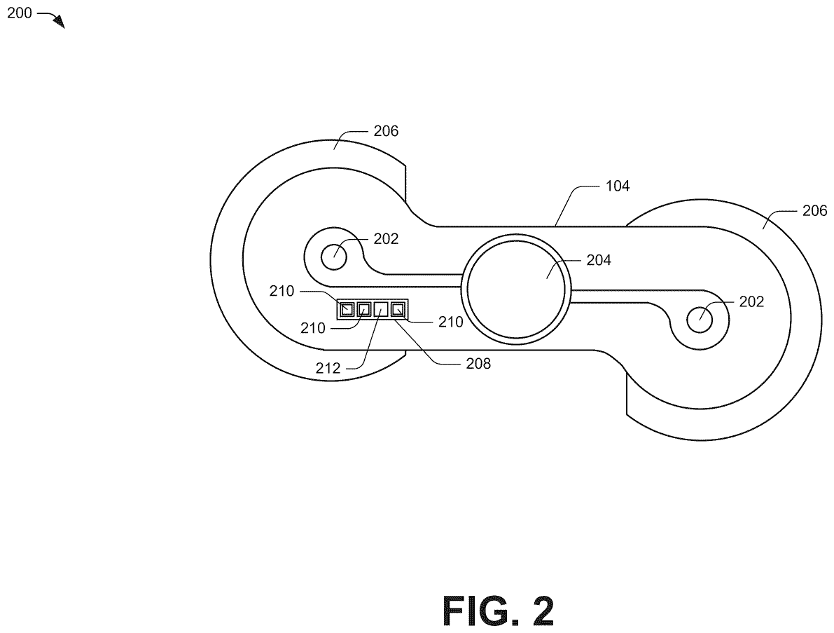

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a PPG sensor (e.g., to measure and record SpO2 and/or produce a photoplethysmogram of the person 102), to name just a few. Certainly, the monitoring device 104 may be configured with any of a variety of types of sensors without departing from the described techniques.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively, or additionally, the monitoring device 104 may have a similar form factor as for clinical settings, but may have different functionality, such as functionality that prevents a wearer from viewing the measurements.

In one or more implementations, the monitoring device 104 may be configured to offload the measurements 108 and/or other data from the monitoring device during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of sleep apnea, for instance.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from storage of the device.

Accordingly, the monitoring device 104 may be configured with one or more ports to enable wired transmission of the measurements to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from local storage of the device to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate one or more predictions 110. For example, the measurements 108 (and/or other measurements such as accelerometer data, PPG data, SpO2 measurements, and so forth) may be processed by a smartphone associated with a user (e.g., the person 102, or an individual associated with the person 102), a smartphone or other dedicated device associated with the monitoring device 104, and/or one or more server computers at a data center or other location that can be utilized by an entity associated with the monitoring device 104, to name just a few. In other words, those other devices may implement at least a portion of the prediction system 114.

In one or more implementations, the monitoring device 104 is configured to transmit the measurements 108 to an external device over a wired connection with the external device, e.g., via USB-C or some other physical, communicative coupling. As used herein, an “external device” is meant to denote a device that is not body-worn. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a healthcare provider, telemedicine service, provider of the monitoring device 104, or medical testing laboratory.

Alternatively, or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with additional (e.g., separate and/or external) devices using one or more wireless communication protocols or techniques. By way of example, the monitoring device 104 may communicate with the additional devices using one or more of Bluetooth® (e.g., Bluetooth® Low Energy links), near-field communication (NFC), Long Term Evolution (LTE™) standards such as 5G, and so forth. The monitoring device 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, and so forth (e.g., an auxiliary or separate monitoring device used in combination with the monitoring device 104). As noted above, examples of such additional measurements include but are not limited to accelerometer data, PPG measurements/signals/waveforms, and/or SpO2 measurements.

In one or more implementations, the analysis platform 106 may be implemented in whole or in part at the monitoring device 104. Alternatively, or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a healthcare provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform 106 includes a storage device 112 and a prediction system 114. In accordance with the described techniques, the storage device 112 is configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a healthcare provider, information about an insurance provider, payment information, prescription information, determined health indicators, account information (e.g., username and password), and so forth. The storage device 112 may also maintain data of other users of a user population.

In the illustrated example 100, the prediction system 114 represents functionality to process the measurements 108 to generate the one or more predictions 110. Alternatively, or in addition, the prediction system 114 may output one or more time sequences indicating an observation or prediction of one or more events over time. It is also to be appreciated that in variations, the prediction system 114 may output different combinations of multiple predictions.

In at least one implementation, the prediction system 114 uses machine learning and/or one or more algorithms to generate the one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively, or additionally, the prediction system 114 may include logic (a machine learning model and/or other types of logic) to pre-process the obtained measurements, such as to extract various cardiovascular and/or other features from the sequences of measurements. The illustrated example 100 also includes one or more predictions 110, which correspond to the output of the prediction system 114.

In the illustrated example 100, the prediction system 114 includes a signal feature correlation algorithm 116. The signal feature correlation algorithm 116 represents functionality to analyze temporal relationships between electrocardiogram (ECG) waveforms and photoplethysmogram (PPG) waveforms collected by the monitoring device 104. In one or more implementations, the signal feature correlation algorithm 116 identifies at least one feature in the ECG waveform, such as an R-wave peak and/or a T-wave peak, and uses the identified at least one feature to locate one or more corresponding features in the PPG waveform based on expected temporal correspondences. For example, a timing of the R-wave peak in the ECG waveform may be used to define a search window for locating a systolic peak in a red PPG waveform and/or an IR PPG waveform. Alternatively, or in addition, a timing of the T-wave peak in the ECG waveform may be used to define a search window for locating a diastolic trough in the red PPG waveform and/or the IR PPG waveform.

The signal feature correlation algorithm 116 enables improved detection of PPG peaks and troughs during low signal-to-noise ratio (SNR) conditions. In scenarios where the red PPG waveform and/or the IR PPG waveform have low amplitude and/or high noise, the signal feature correlation algorithm 116 may leverage timing information from the ECG waveform to more accurately detect the peaks and troughs in the red PPG waveform and/or the IR PPG waveform. This approach may be used to calculate SpO2 values with improved accuracy, as SpO2 calculations rely on accurate identification of peaks and troughs in the red and/or IR PPG waveforms. In some implementations, the signal feature correlation algorithm 116 may calibrate timing offsets between ECG and PPG signals. In one or more implementations, the signal feature correlation algorithm 116 may compare the ECG waveform to a high-SNR segment of the red PPG waveform and/or the IR PPG waveform to determine a timing offset between the ECG waveform and the red PPG waveform and/or the IR PPG waveform. Once the timing offset is determined, the signal feature correlation algorithm 116 may apply the calibrated offset when using ECG features to locate features in the red PPG waveform and/or the IR PPG waveform during subsequent low-SNR conditions. Alternatively, or in addition, in an example where there is a timing difference between the PPG sensor and the ECG sensor, the PPG sensor may be used to calibrate a timing of the ECG sensor using a high-SNR segment of the red PPG waveform and/or the IR PPG waveform. Accordingly, the signal feature correlation algorithm 116 may calibrate timing offsets bidirectionally between the ECG waveform and the PPG waveforms, at least in some examples.

In some implementations, the monitoring device 104 may include a third light source configured to emit light at a wavelength that provides a higher SNR relative to red light and the IR light, such as green light. The third PPG waveform may have higher signal amplitude and may be less susceptible to noise, which may be used to establish timing calibration between the ECG waveform and the red and/or IR PPG waveforms. By way of example, the signal feature correlation algorithm 116 may use the third PPG waveform to calibrate the timing offset and may further apply the calibrated offset when locating features in the red PPG waveform and/or the IR PPG waveform during low-SNR conditions.

In this way, the signal feature correlation algorithm 116 enables improved SpO2 measurement by leveraging ECG timing information to enhance feature detection in the red PPG waveform and/or the IR PPG waveform, particularly during conditions where the red PPG waveform and/or the IR PPG waveform have low amplitude or high noise, without increased LED current or additional light sources that would consume more power.

FIG. 2 depicts a non-limiting example 200 of a monitoring device. The illustrated example 200 depicts the monitoring device 104.

In accordance with the described techniques, the monitoring device 104 includes one or more sensors 202, examples of which include, but are not limited to, one or more pairs of electrodes, an accelerometer, a PPG sensor, temperature sensor(s), and sweat sensors, to name just a few. The monitoring device 104 may also include a transmitter 204, which may be enclosed in a housing, for example. In this example 200, the monitoring device 104 further includes one or more adhesive portions 206 configured as attachment components. In operation, the monitoring device 104 is configured to be applied to the skin via the one or more adhesive portions 206, such that, for example, the one or more sensors 202 are positioned to detect and record the electrical activity of the heart of the person 102, e.g., to produce an electrocardiogram (ECG or EKG). In at least one implementation, the monitoring device 104 may be removed by peeling the one or more adhesive portions 206 from the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the electrical activity of the heart. In one or more implementations, the processor further generates one or more communicable packages of data that include one or more of the measurements 108 and/or other measurements, such as accelerometer data, PPG data, and/or oxygen saturation (SpO2) measurements. Alternately or additionally, the processor produces and/or causes storage of other data, which may be used for predicting classifications of sleep apnea.

In implementations where the monitoring device 104 is configured for wireless transmission, the transmitter 204 may transmit the measurements wirelessly as a stream of data to a computing device (e.g., the analysis platform 106). In one or more implementations, for instance, the monitoring device 104 is configured to transfer (e.g., transmit and/or receive) information (e.g., ECG and/or PPG measurements) via a Bluetooth® Low Energy (BLE) connection. Alternatively, or additionally, the monitoring device 104 may buffer the measurements 108 (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

In this example, the monitoring device 104 is depicted as including an optical sensor 208. In some implementations, the optical sensor 208 may include one or more light-emitting components and one or more light-detecting components configured to obtain PPG measurements in one or more wavelength channels. For example, the optical sensor 208 includes one or more light sources 210, such as light-emitting diodes (LEDs) and/or laser diodes. The one or more light sources 210, for instance, may emit light within the red to infrared spectrum, which penetrates the skin and underlying tissues more efficiently than light having shorter wavelengths (e.g., light within the ultraviolet to orange regions of the spectrum). By way of example, a given light source of the one or more light sources 210 may emit light at a wavelength that is between approximately 600 nanometers (nm) and approximately 1000 nm. In at least one variation, however, the one or more light sources 210 of the optical sensor 208 emit light of a shorter wavelength (e.g., green light) in addition to or as an alternative to the longer wavelength light. In one or more further variations, at least one of the one or more light sources 210 emits light of a near-IR (NIR) wavelength. In one or more implementations, the optical sensor 208 is configured to emit and detect multiple different wavelengths of light to capture different physiological parameters. In the example 200, the optical sensor 208 is shown having three light sources 210. However, it is to be appreciated that the optical sensor 208 may have more than three light sources 210 or fewer than three light sources 210 in variations.

The optical sensor 208 may also include at least one photodetector 212, such as a photodiode, positioned to detect light reflected from or transmitted through tissue. Although one photodetector 212 is shown in FIG. 2, it is to be appreciated that the optical sensor 208 may include multiple photodetectors in variations. As the heart pumps blood through the body, the volume of blood in the microvascular bed of the tissue fluctuates. As blood volume in the tissue changes with each heartbeat, the amount of light absorbed or reflected may vary, allowing the optical sensor 208 to detect pulsatile blood flow. By way of example, the at least one photodetector 212 detects these changes in the light absorbed or reflected, resulting in the PPG signal. The PPG signal may be used to derive various physiological parameters, such as pulse rate, blood oxygen saturation (SpO2), and/or respiratory rate. By way of example, as oxygenated and deoxygenated hemoglobin absorb different amounts of red and infrared light, a ratio of absorbed light at different wavelengths can be used to calculate the oxygen saturation of the blood. The optical sensor 208 may take these measurements continuously or at regular intervals, providing a stream of SpO2 data. This data may be processed by the monitoring device 104 or transmitted to the analysis platform 106 for further analysis.

It is to be appreciated that the placement of the optical sensor 208 shown in the example 200 of FIG. 2 is for illustration and not limitation, and variations exist. Moreover, it is to be understood that the optical sensor 208 is positioned on an underside of the monitoring device 104 to contact the skin of the person 102 and/or direct light toward the skin of the person 102.

The optical sensor 208 may include a lens. In some implementations, the lens may have one or more of a convex configuration, a flat configuration, a domed configuration, or a concave configuration. The specific lens configuration may be selected based on the desired optical properties and performance characteristics of the optical sensor 208.

In one or more implementations, the monitoring device 104 may combine PPG sensing with other modalities, such as ECG or accelerometry, to provide a more comprehensive picture of the physiological state of the person 102. This multi-modal approach may enhance the ability of the monitoring device 104 to detect and monitor various health conditions, including sleep disorders, arrhythmias, or changes in cardiovascular function. In at least one variation, the optical sensor 208 is located on a separate device from the one or more sensors 202, such as an auxiliary device and/or a second monitoring device.

### Physiological Signal Detection and Correlation

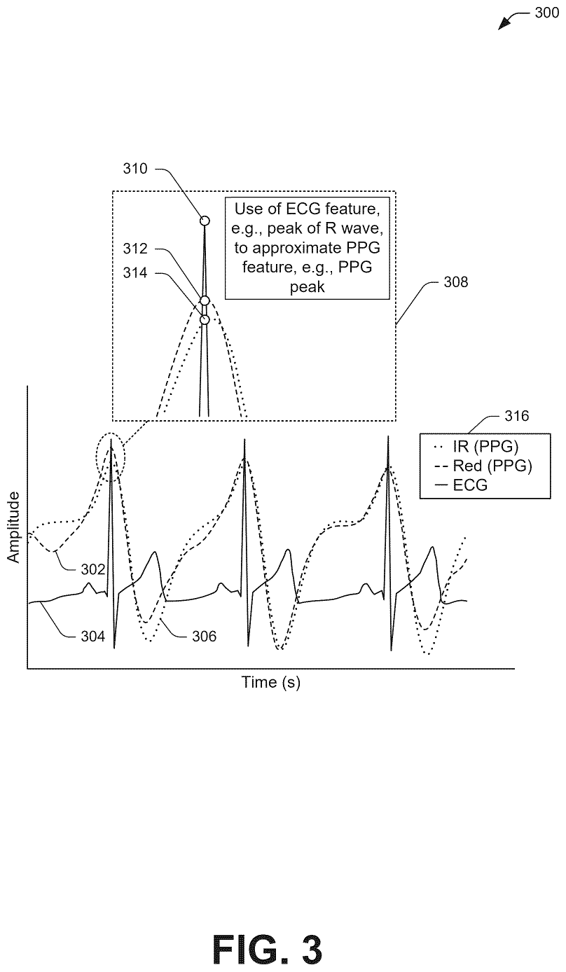

FIG. 3 depicts a first waveform diagram 300 illustrating a temporal relationship between electrocardiogram (ECG) and photoplethysmogram (PPG) signals, such as recorded by the monitoring device 104. The first waveform diagram 300 includes a red PPG waveform 302 (e.g., a PPG waveform of a red wavelength channel), an ECG waveform 304, and an infrared (IR) waveform 306 (e.g., a PPG waveform of an IR wavelength channel) plotted against time. A magnified view 308 highlights a region where an ECG peak 310 aligns with a red PPG peak 312 and an IR PPG peak 314, demonstrating a temporal correspondence between these physiological signals. A legend 316 in the first waveform diagram 300 identifies the red PPG waveform 302 as a dashed line, the ECG waveform 304 as a solid line, and the IR waveform 306 as a dotted line.

The first waveform diagram 300 may be generated based on physiological signals collected by the monitoring device 104. In some cases, the monitoring device 104 includes multiple sensors for collecting different types of physiological measurements. For example, the one or more sensors 202 of the monitoring device 104 (e.g., a first sensor) may collect a first physiological signal corresponding to a first physiological measurement, such as the ECG waveform 304. The optical sensor 208 of the monitoring device 104 (e.g., a second sensor) may collect a second physiological signal corresponding to a second physiological measurement, such as the red PPG waveform 302 and/or the IR waveform 306.

The analysis platform 106 may process these collected signals to detect features and/or temporal relationships between the different physiological measurements. By way of example, the signal feature correlation algorithm 116 of the prediction system 114 may detect a feature of the first physiological signal and may further detect a feature of the second physiological signal based on a timing of the feature of the first physiological signal. A variety of features are considered, including but not limited to peaks, troughs, slopes, amplitudes, intervals, and waveform morphologies. In some implementations, the features include R-wave peaks, T-wave peaks, QRS complexes, P-waves, and/or additional elements present in ECG signals. For PPG signals, the features may include systolic peaks, diastolic troughs, pulse amplitudes, pulse transit times, and so forth. This is by way of example and not limitation, and a variety of features of the first physiological signal and the second physiological signal are considered.

In the illustrated example, the feature of the first physiological signal includes the ECG peak 310 in the ECG waveform 304. The ECG peak 310, for instance, corresponds to a peak of an R-wave in the ECG measurement. Based on an expected temporal correspondence between the feature of the first physiological signal and the second physiological measurement, the signal feature correlation algorithm 116 of the prediction system 114 may detect a feature of the second physiological signal. For example, the red PPG peak 312 in the red PPG waveform 302 and/or the IR PPG peak 314 in the IR waveform 306 may be identified based on their temporal alignment with the ECG peak 310, plus a calibrated offset value if applicable. The ECG peak 310, for instance, may be used as a starting point to define a search window for locating the peaks in the red PPG waveform 302 and the IR waveform 306. In one or more implementations, the search window is a time interval that is centered on or offset from (e.g., based on the calibrated offset value) the timing of the ECG peak 310 and extends for a predetermined duration, such as 100 to 300 milliseconds. By using the search window, the signal feature correlation algorithm 116 may detect local maxima within the constrained time interval rather than across the entire PPG waveform, which reduces the likelihood of false detections caused by noise spikes or artifacts occurring outside the expected timing range.

In one or more implementations, the features of the second physiological signal may be identified using various techniques and approaches, including but not limited to: signal processing algorithms to detect a local maximum and/or a local minimum in the various waveforms, derivative-based methods to detect inflection points and/or rapid changes, template matching techniques, adaptive thresholding algorithms, machine learning models such as convolutional or recurrent neural networks, frequency domain analysis techniques such as wavelet transforms or Fourier analysis, multi-signal fusion techniques combining information from multiple channels, adaptive filtering techniques, contextual information adjustments, ensemble methods combining multiple feature detection algorithms, and so forth.

The temporal relationship illustrated in the first waveform diagram 300 demonstrates that features in the ECG waveform 304 are usable to enhance detection of corresponding features in the PPG signals. This approach may be particularly useful when the PPG signals have a low signal-to-noise ratio that makes it challenging to accurately detect peaks and troughs solely based on the PPG waveforms. This approach may also reduce power consumption relative to using a third PPG wavelength (e.g., green) for the same purpose.

In some cases, the prediction system 114 may use the identified features to calculate various physiological parameters. For example, time intervals between successive ECG peaks 310 may be used to determine heart rate. Additionally, amplitudes and timing of the red PPG peaks 312 and/or IR PPG peaks 314 may be used in calculations of SpO2, as described in more detail below.

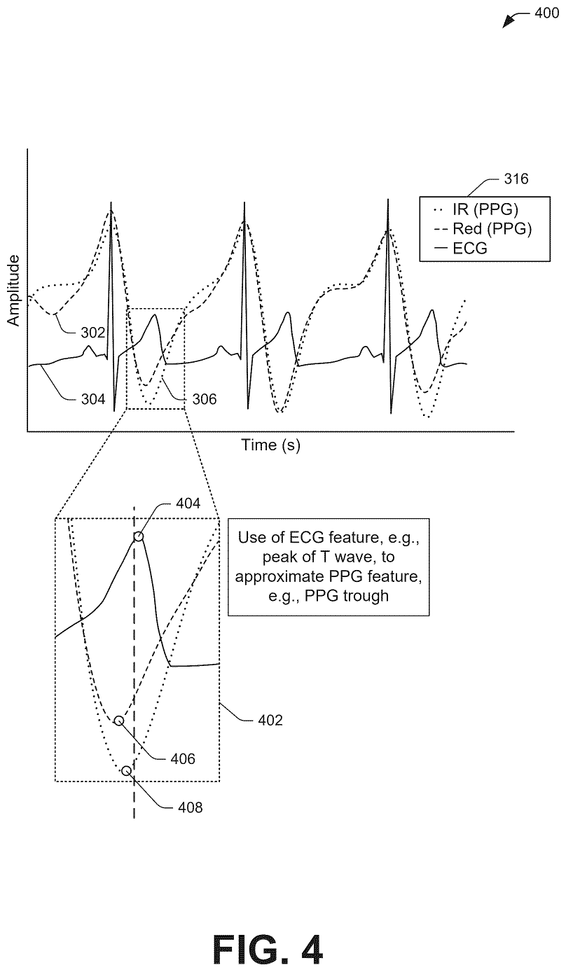

FIG. 4 depicts a second waveform diagram 400 illustrating a temporal relationship between electrocardiogram (ECG) and photoplethysmogram (PPG) signals. The second waveform diagram 400 includes the red PPG waveform 302, the ECG waveform 304, the IR waveform 306 plotted against time, and the legend 316. A magnified view 402 highlights a region where a T-wave peak 404 in the ECG waveform 304 aligns with a red PPG signal trough 406 and an IR PPG signal trough 408, demonstrating a temporal correspondence between these physiological signals.

The second waveform diagram 400 may be generated based on physiological signals recorded by the monitoring device 104. In some examples, the analysis platform 106 processes these collected signals to detect features and temporal relationships between the different physiological measurements. By way of example, the signal feature correlation algorithm 116 of the prediction system 114 may identify, extract, and/or detect a feature of the first physiological signal, such as the T-wave peak 404 in the ECG waveform 304. Based on an expected temporal correlation between the feature of the first physiological signal and the second physiological signal, the signal feature correlation algorithm 116 may identify, extract, and/or detect a feature of the second physiological signal. For example, the red PPG signal trough 406 in the red PPG waveform 302 and/or the IR PPG signal trough 408 in the IR waveform 306 may be identified based on their temporal alignment with the T-wave peak 404, plus a calibrated offset value if applicable. The T-wave peak 404, for instance, may serve as a starting point to define a search window for locating the troughs in the red PPG waveform 302 and the IR waveform 306. In one or more implementations, the search window is a time interval that is centered on or offset from (e.g., based on the calibrated offset value) the timing of the T-wave peak 404 and extends for a predetermined duration, such as 100 to 300 milliseconds. By using the search window, the signal feature correlation algorithm 116 may identify, extract, and/or detect local minima within the constrained time interval rather than across the entire PPG waveform, which reduces the likelihood of false detections for more accurate detection of the red PPG signal trough 406 and/or the IR PPG signal trough 408.

For instance, a dashed vertical line in the magnified view 402 represents a timing reference point derived from the T-wave peak 404, e.g., as offset based on the calibrated offset value mentioned above. In this example, the timing reference point is closer to the red PPG signal trough 406 and the IR PPG signal trough 408 than the T-wave peak 404. The search window may be centered on the timing reference point, for example, enabling the red PPG signal trough 406 and the IR PPG signal trough 408 to be efficiently located.

In some cases, the prediction system 114 may use the identified features to calculate various physiological parameters. For example, the prediction system 114 may use the detected peaks and troughs of the PPG signal to calculate SpO2 values. For instance, the prediction system 114 may analyze the red PPG waveform 302 and the IR waveform 306 to determine a ratio of oxygenated to deoxygenated hemoglobin in the blood. In this way, the techniques described herein support accurate determination of a variety of physiological measurements.

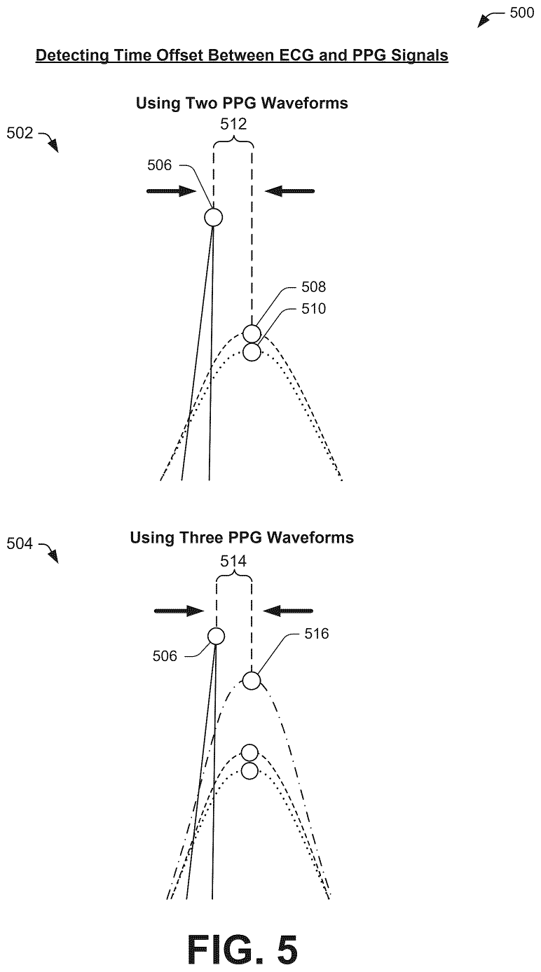

FIG. 5 depicts a diagram 500 illustrating the detection of time offsets between ECG and PPG signals. The diagram 500 includes a first waveform plot 502 and a second waveform plot 504 that demonstrate different examples of signal measurements.

In the first waveform plot 502, an ECG peak 506 is shown alongside a red PPG signal peak 508 and an IR PPG signal peak 510. A first time offset 512 is indicated between the ECG peak 506 and the PPG signal peaks. The first time offset 512 may represent a temporal delay between the electrical activity of the heart, as detected by ECG electrodes of the one or more sensors 202, and corresponding blood volume changes detected by the optical sensor 208. By way of example, the first time offset 512 may be due to biological factors such as pulse transit time, sensor design, sensor placement, and so forth.

The second waveform plot 504 displays the ECG peak 506 with an additional PPG signal peak 516. In some cases, the additional PPG signal peak 516 may be generated by an additional light source of the optical sensor 208. For example, the additional light source may emit light at a wavelength that provides a higher signal-to-noise ratio (SNR), e.g., a higher fidelity waveform, compared to the red and IR wavelengths typically used for SpO2 measurements. The second waveform plot 504 also shows a second time offset 514 between the signals.

The detection and analysis of the first time offset 512 and the second time offset 514 may be performed by the signal feature correlation algorithm 116 of the prediction system 114. In some cases, the signal feature correlation algorithm 116 may use the time offsets to calibrate a timing between the ECG and PPG signals. This calibration process may involve adjusting a relative timing of the signals to account for physiological delays and sensor placement variations. In one or more implementations, the calibration may use a high-SNR segment of the red PPG waveform and/or the IR PPG waveform to determine the timing offset.

In one or more implementations, the monitoring device 104 and/or the analysis platform 106 may apply noise filtering to the ECG signal and/or the PPG signal(s) before, after, and/or during time offset analysis. The noise filtering may help improve an accuracy of peak detection and time offset measurements. In one or more implementations, the signal feature correlation algorithm 116 may evaluate a consistency of detected peaks and troughs and assess signal quality metrics to ensure reliable calibration, such as via implementation of one or more thresholds related to signal quality to perform various actions. For example, the one or more thresholds may include a signal-to-noise ratio threshold, an amplitude threshold, a peak consistency threshold, and/or a time offset variability threshold. The various actions may include discarding or excluding a low-quality signal segment from calibration, widening or narrowing a search window for feature detection, flagging a measurement as unreliable, triggering recalibration of the timing offset, adjusting a confidence level for a calculated parameter such as an SpO2 value, and/or requesting an additional signal sample. By way of example, the signal feature correlation algorithm 116 may compare a signal-to-noise ratio of a PPG signal segment to the signal-to-noise ratio threshold and, responsive to the signal-to-noise ratio being below the signal-to-noise ratio threshold, exclude the PPG signal segment from calibration. As another example, the signal feature correlation algorithm 116 may widen the search window for feature detection responsive to a peak consistency metric falling below the peak consistency threshold. This is by way of example and not limitation, and a variety of thresholds and actions are considered.

The calibrated timing information may be used to enhance an accuracy of SpO2 calculations. For example, the signal feature correlation algorithm 116 may use the calibrated timing to precisely locate PPG peaks and/or troughs, even in low SNR conditions. This supports robust SpO2 determination relative to conventional techniques that rely solely on PPG signal analysis.

In some implementations, the signal feature correlation algorithm 116 may continuously monitor the signals and update the calibration based on changing signal quality and/or other conditions. This adaptive approach may help maintain measurement accuracy over time and across different physiological states of the person 102. Furthermore, when outputting and/or reporting SpO2 values, the signal feature correlation algorithm 116 may also provide a signal quality indicator. The signal quality indicator may be based on factors such as a consistency of detected peaks and troughs, a magnitude of the time offsets, and/or other signal quality metrics. The signal quality indicator may help users and healthcare providers assess the reliability of the reported SpO2 values. By leveraging the temporal relationships between ECG and PPG signals, the techniques described herein provide accurate and reliable physiological measurements, particularly in challenging monitoring conditions.

The following discussion describes techniques that are implementable utilizing the previously described systems and devices. Aspects of each of the procedures (e.g., methods) can be implemented in hardware, firmware, software, or a combination thereof. The procedures are shown as a set of blocks that specify operations that can be performed by one or more devices and are not necessarily limited to the orders shown for performing the operations by the respective blocks. One or more blocks of the procedures, for instance, specify operations that can be programmable by hardware (e.g., processor, microprocessor, controller, firmware) as executable instructions, thereby creating a special-purpose machine for carrying out an algorithm as illustrated by the flow diagram. As a result, the instructions are storable on a non-transitory computer-readable storage medium that causes the hardware to perform the algorithm. In portions of the following discussion, reference will be made to FIGS. 1-5.

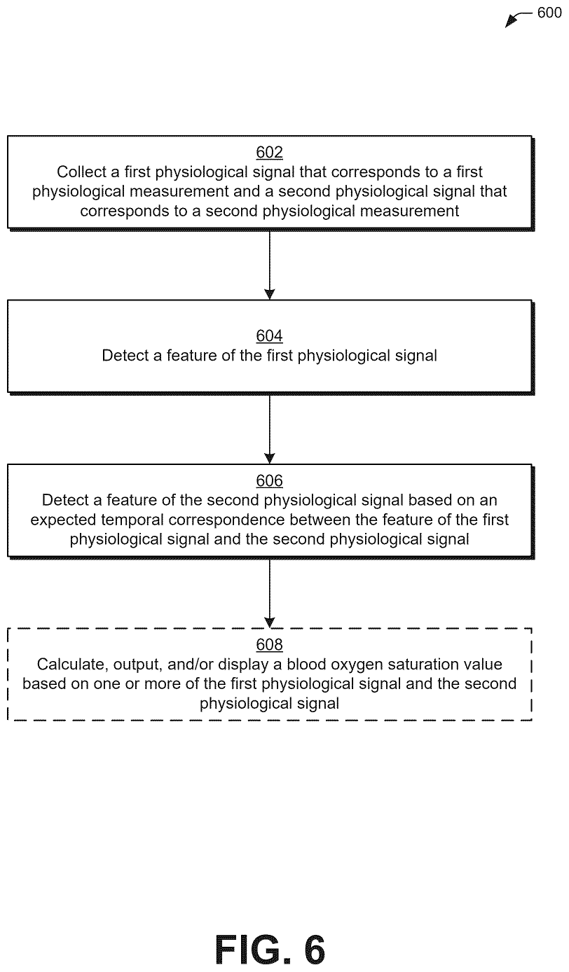

FIG. 6 illustrates a method 600 for detecting features in physiological signals using cross-signal temporal correlations.

A first physiological signal corresponding to a first physiological measurement and a second physiological signal corresponding to a second physiological measurement are collected (block 602). By way of example, the monitoring device 104 may collect the first physiological signal and the second physiological signal. The first physiological measurement may be an electrocardiogram (ECG) measurement, and the first physiological signal may include an ECG waveform collected by ECG electrodes of the monitoring device 104. The second physiological measurement may be a photoplethysmogram (PPG) measurement, and the second physiological signal may include a PPG waveform, such as the red PPG waveform 302 and/or the IR waveform 306, collected by the optical sensor 208 of the monitoring device 104.

A feature of the first physiological signal is detected (block 604). By way of example, the signal feature correlation algorithm 116 of the prediction system 114 may detect the feature of the first physiological signal (e.g., a first feature), such as the ECG peak 310 in the ECG waveform 304. The ECG peak 310 may correspond to an R-wave peak of the ECG waveform 304. In one or more implementations, the signal feature correlation algorithm 116 may additionally or alternatively detect the T-wave peak 404 in the ECG waveform 304 or another feature of the ECG waveform 304.

A feature of the second physiological signal is detected based on an expected temporal correspondence between the feature of the first physiological signal and the second physiological signal (block 606). By way of example, the signal feature correlation algorithm 116 may detect the feature of the second physiological signal (e.g., a second feature that is different from the first feature), such as the red PPG peak 312 in the red PPG waveform 302 and/or the IR PPG peak 314 in the IR waveform 306, based on a temporal alignment with the ECG peak 310. For instance, the signal feature correlation algorithm 116 may use a timing of the ECG peak 310, plus a calibrated offset if applicable, as a starting point to define a search window for locating the red PPG peak 312 and/or the IR PPG peak 314. Alternatively, or in addition, the signal feature correlation algorithm 116 may detect the red PPG signal trough 406 in the red PPG waveform 302 and/or the IR PPG signal trough 408 in the IR waveform 306 based on a temporal alignment with the T-wave peak 404.

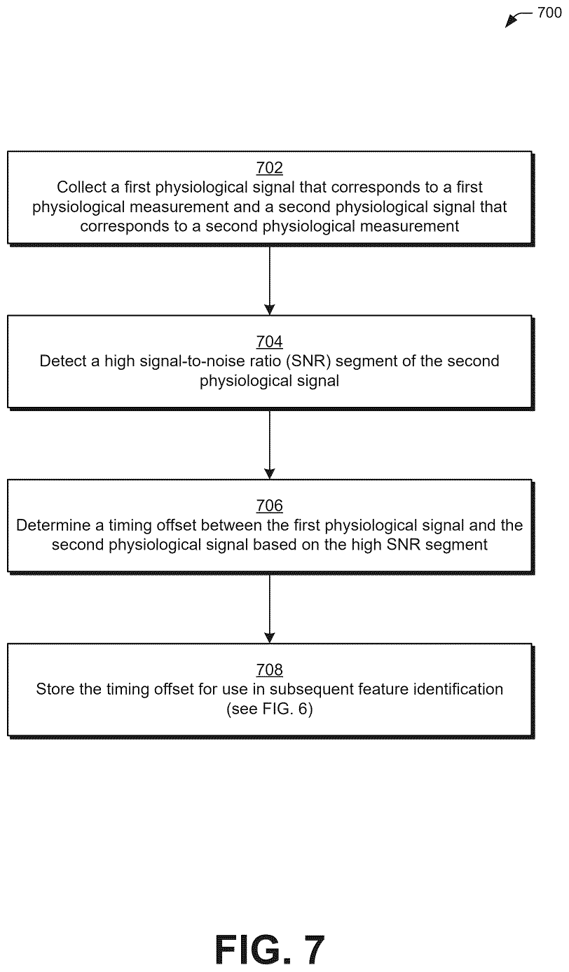

In one or more implementations, the method 600 may be performed in conjunction with a calibration method, such as will be described below with reference to FIG. 7, to determine the calibrated offset between the ECG and PPG signals.

In some cases, a blood oxygen saturation value is calculated, output, and/or displayed based on one or more of the first physiological signal and the second physiological signal (block 608). For instance, the prediction system 114 may calculate AC and DC components for the PPG waveform, e.g., the red PPG waveform 302 and/or the IR waveform 306. The AC component may be calculated as a difference between the peak and trough values of the respective waveforms. The DC component may be calculated using the trough value of the respective waveforms.

The prediction system 114 may compute a ratio of ratios using the calculated AC and DC components. For example, the ratio of ratios may be computed as:

$$R = \frac{{AC}_{red} / {DC}_{red}}{{AC}_{IR} / {DC}_{IR}}$$

where AC_{red} and DC_{red} are the AC and DC components of the red PPG waveform 302, and AC_{IR} and DC_{IR} are the AC and DC components of the IR waveform 306.

The prediction system 114 may use a calibration curve to convert the computed ratio of ratios to a blood oxygen saturation (SpO2) value. In one or more implementations, the method 600 may include outputting the blood oxygen saturation value for display by a display device. For example, the prediction system 114 may send the calculated SpO2 value to a display device of the monitoring device 104 or to an external display device for presentation to the person 102 and/or a healthcare provider.

In this way, the method 600 leverages temporal correlations between ECG and PPG signals to locate one or more features in the PPG waveforms, which may enable accurate feature detection even when the red PPG waveform 302 and/or the IR waveform 306 have a low signal-to-noise ratio. This approach may also reduce power consumption relative to using a third PPG wavelength for the same purpose, which may extend a wear time of the monitoring device 104.

FIG. 7 illustrates a method 700 for calibrating a timing offset between physiological signals.

A first physiological signal corresponding to a first physiological measurement and a second physiological signal corresponding to a second physiological measurement are collected (block 702). In some cases, the monitoring device 104 may collect the first physiological signal and the second physiological signal. For example, the first physiological measurement may be an ECG measurement, and the first physiological signal may include an ECG waveform collected by ECG electrodes of the monitoring device 104. The second physiological measurement may be a PPG measurement, and the second physiological signal may include a PPG waveform, such as the red PPG waveform 302 and/or the IR waveform 306, collected by the optical sensor 208 of the monitoring device 104.

A high signal-to-noise ratio (SNR) segment of the second physiological signal is detected (block 704). By way of example, the prediction system 114 may detect the high-SNR segment based on signal quality metrics. The signal quality metrics may include a signal amplitude, a noise level, a consistency of detected features, a peak-to-peak amplitude variability, a baseline stability, and/or an absence of motion artifacts. In one or more implementations, the signal feature correlation algorithm 116 may compare the signal quality metrics to one or more thresholds to determine whether a segment of the second physiological signal is a high-SNR segment. For instance, the signal feature correlation algorithm 116 may indicate a segment is a high-SNR segment responsive to the signal amplitude exceeding an amplitude threshold and/or the noise level falling below a noise threshold. Alternatively, or in addition, the signal feature correlation algorithm 116 may evaluate multiple consecutive cardiac cycles within the segment to assess consistency of detected features, such as consistency of peak timing and/or peak amplitude across the consecutive cardiac cycles.

A timing offset between the first physiological signal and the second physiological signal is determined based on the high-SNR segment (block 706). By way of example, the signal feature correlation algorithm 116 may compare a feature of the first physiological signal, such as the ECG peak 506, to a corresponding feature of the second physiological signal, such as the red PPG signal peak 508 and/or the IR PPG signal peak 510, within the high-SNR segment to determine the timing offset (e.g., the first time offset 512 of the first waveform plot 502). Alternatively, or in addition, the optical sensor 208 may include an additional light source configured to emit light at a wavelength that provides a higher SNR relative to the red and IR wavelengths, such as green light. Therefore, in one or more implementations, the signal feature correlation algorithm 116 may use a PPG waveform generated by the additional light source to determine the timing offset, such as in the additional PPG signal peak 516 of the second waveform plot 504.

The timing offset is stored for use in subsequent feature identification (block 708). By way of example, the signal feature correlation algorithm 116 may store the timing offset, such as the first time offset 512 and/or the second time offset 514, in the storage device 112. The stored timing offset may be applied when using ECG features to locate features in the red PPG waveform 302 and/or the IR waveform 306 when subsequent measurements are obtained, as described above with reference to the method 600.

In one or more implementations, the method 700 may be performed periodically or continuously to update the calibrated timing offset based on changing conditions, such as due to a change in sensor placement, a physiological state of the person 102, and/or signal quality.

In this way, the method 700 calibrates a timing relationship between ECG and PPG signals, which enables the signal feature correlation algorithm 116 to accurately locate PPG features based on ECG timing information, even during low-SNR conditions. This calibration approach may improve an accuracy of physiological parameter calculations, such as SpO2, and may reduce power consumption relative to using a third PPG wavelength for the same purpose.

### Machine Learning and AI in Physiological Signal Detection and Correlation

The previous examples describe various instances of artificial intelligence (“AI”) models or machine learning models, such as with respect to the prediction system 114 and the signal feature correlation algorithm 116. In one or more examples, an AI model, e.g., a machine learning model, refers to a computer representation that is tunable (e.g., through training and retraining) based on inputs to approximate unknown functions, automatically and without user intervention, without being actively programmed by a user. For instance, the term “machine learning model” includes a model that utilizes algorithms to learn from, and make predictions on, known data by analyzing training data to learn and relearn to generate outputs that reflect patterns and attributes of the training data.

In the context of physiological signal detection and correlation, machine learning models are implementable (e.g., by one or more processing devices of the analysis platform 106) to analyze physiological data patterns such as to detect features in ECG waveforms, detect corresponding features in PPG waveforms, determine timing offsets between signals, and/or calculate physiological parameters such as blood oxygen saturation (SpO2). For example, the prediction system 114 and the signal feature correlation algorithm 116 may each utilize one or more machine learning models to process physiological data such as the ECG waveform 304, the red PPG waveform 302, the IR waveform 306, and/or other measurements collected by the monitoring device 104. Examples of machine learning models applicable to physiological signal correlation and feature detection include neural networks, convolutional neural networks (CNNs) (e.g., for analyzing waveform data and detecting patterns and/or features indicative of ECG peaks, PPG peaks, and PPG troughs), long short-term memory (LSTM) neural networks (e.g., to analyze temporal physiological patterns and detect temporal correspondences between ECG and PPG signals), generative adversarial networks (GANs), decision trees (e.g., for classification of signal quality conditions), support vector machines, linear regression, logistic regression for signal quality assessment, Bayesian networks, random forest learning for feature importance in physiological signal correlation, dimensionality reduction algorithms, boosting algorithms, deep learning neural networks, and so forth.

A machine learning model, for instance, is configurable using a plurality of layers having, respectively, a plurality of nodes. The plurality of layers is configurable to include an input layer, an output layer, and one or more hidden layers. In the context of physiological signal detection and correlation, the input layer may receive various physiological parameters from the measurements 108, such as ECG features including R-wave peaks and T-wave peaks, PPG waveform data from the red PPG waveform 302 and the IR waveform 306, timing information, signal amplitude data, and signal quality metrics. The hidden layers, for instance, process these inputs through weighted connections to detect complex patterns indicative of temporal correspondences between ECG and PPG signals, e.g., patterns that are not detectable using conventional threshold-based methods. The output layer may produce feature identifications such as the ECG peak 310, the red PPG peak 312, the IR PPG peak 314, the T-wave peak 404, the red PPG signal trough 406, and the IR PPG signal trough 408, timing offset values such as the first time offset 512 and the second time offset 514, signal quality assessments, or generate the one or more predictions 110 that incorporate correlated physiological signal analysis. Calculations are performed by the nodes within the layers via hidden states through a system of weighted connections that are “learned” during training of the machine learning model to implement a variety of physiological signal correlation and feature detection tasks.

In order to train the machine learning model for physiological signal detection and correlation, training data are received that provide examples of “what is to be learned” by the machine learning model, i.e., as a basis to learn patterns and/or features from the data. For signal correlation applications, the training data may include labeled datasets of physiological measurements with known feature locations, such as ECG waveforms with annotated R-wave peaks and T-wave peaks, PPG waveforms with annotated systolic peaks and diastolic troughs, timing offset data between ECG and PPG signals with verified temporal correspondences, and/or physiological data with labeled signal quality conditions. A machine learning system that includes the machine learning model, for instance, collects and preprocesses the training data that include input features (e.g., ECG waveforms, PPG waveforms, signal amplitude data, noise levels) and corresponding target labels (e.g., feature locations, timing offsets, signal quality classifications, or SpO2 values).

The machine learning system is further operable to initialize various parameters of the machine learning model, which are usable by the machine learning model as internal variables to represent and process information during training. These parameters are further usable to represent inferences gained through training. In one or more implementations, the training data are separated into batches to improve processing and optimization efficiency of the parameters of the machine learning model during training, which may be beneficial for model accuracy when processing large volumes of physiological time-series data from multiple sensors with varying signal-to-noise ratios and temporal characteristics.

The training data are then received by the machine learning model as inputs and used to generate predictions based on a current state of parameters of layers and corresponding nodes of the model, a result of which is output as output data, e.g., a feature location, timing offset value, signal quality classification, SpO2 value, or the like. For example, the analysis platform 106 includes a machine learning model that is trained to recognize patterns and/or features in physiological data that correlate ECG features with PPG features, which enables the signal feature correlation algorithm 116 to accurately detect PPG peaks and troughs based on ECG timing information and the prediction system 114 to calculate accurate SpO2 values even during low signal-to-noise ratio conditions.

Training of the machine learning model can include calculation of a loss function to quantify a loss associated with operations performed by nodes of the machine learning model. The loss function is configurable in various ways to control operation or functionality of the machine learning model. For instance, the loss function may be designed to prioritize accuracy in feature detection while minimizing false identifications that could lead to inaccurate SpO2 calculations. Calculation of the loss function, for instance, includes comparing a difference between predictions specified in the output data (e.g., predicted feature locations or timing offsets) with target labels specified by the training data (e.g., verified ground truth feature locations). The loss function is configurable in a variety of ways, examples of which include regret, quadratic loss function as part of a least squares technique for continuous timing offset parameters, cross-entropy loss for signal quality classification tasks, custom loss functions that incorporate power efficiency requirements or accuracy priorities specific to particular physiological monitoring applications, and so forth.

The training data are usable to support a variety of usage scenarios in physiological signal detection and correlation. For example, the machine learning model can be trained to detect specific patterns in physiological data (e.g., ECG and PPG data) that enable accurate feature correlation, detect temporal correspondences between ECG R-wave peaks and PPG systolic peaks, recognize temporal correspondences between ECG T-wave peaks and PPG diastolic troughs, or detect subtle signal quality changes that may improve timing offset calibration precision. The models can be configured to operate within computational constraints of real-time signal processing while providing accurate feature detection decisions. The models can further be reconfigured, e.g., with expanded capabilities, for more sophisticated physiological signal analysis when processing historical data or performing detailed diagnostic assessments. This adaptive approach enables efficient use of computational resources devoted to machine learning processes while ensuring comprehensive physiological signal correlation capabilities are available when needed for accurate SpO2 calculation across varying signal quality conditions.

It should be understood that many variations are possible based on the disclosure herein. Although features and elements are described above in particular combinations, each feature or element is usable alone without the other features and elements or in various combinations with or without other features and elements.

What is claimed is:

1. A method for temporal correlation of physiological signals, comprising:
collecting a first physiological signal that corresponds to a first physiological measurement and a second physiological signal that corresponds to a second physiological measurement;
detecting a first feature of the first physiological signal; and
detecting a second feature of the second physiological signal based on an expected temporal correspondence between the first feature and the second physiological signal.

2. The method of claim 1, wherein the first physiological signal is collected from a first sensor of a wearable device, and the second physiological signal is collected by a second sensor of the wearable device or a separate wearable device.

3. The method of claim 1, wherein the first physiological signal is an electrocardiogram (ECG) waveform corresponding to an ECG measurement, and the second physiological signal is a photoplethysmogram (PPG) waveform corresponding to a PPG measurement.

4. The method of claim 3, wherein the PPG waveform includes one or more of an infrared (IR) wavelength channel or a red wavelength channel.

5. The method of claim 3, wherein the first feature is an ECG feature of the ECG waveform, and the second feature is a PPG feature of the PPG waveform.

6. The method of claim 5, wherein the ECG feature includes an R-wave peak, and the PPG feature includes a systolic peak.

7. The method of claim 5, wherein the ECG feature includes a T-wave peak, and the PPG feature includes a diastolic trough.

8. The method of claim 3, wherein the first feature includes one or more of a peak, a trough, a complex, or an interval of the ECG waveform, and the second feature includes one or more of a PPG peak or a PPG trough of the PPG waveform.

9. The method of claim 1, further comprising:
calculating a blood oxygen saturation value based at least on the second feature.

10. The method of claim 1, further comprising calibrating a timing offset between the first physiological signal and the second physiological signal.

11. The method of claim 10, wherein calibrating the timing offset comprises:
detecting a high signal-to-noise ratio segment of the second physiological signal;
determining the timing offset between the first physiological signal and the second physiological signal based on the high signal-to-noise ratio segment; and
detecting the second feature further based on the timing offset.

12. A system for temporal correlation of physiological signals, comprising:
a first sensor configured to collect a first physiological signal corresponding to a first physiological measurement;
a second sensor configured to collect a second physiological signal corresponding to a second physiological measurement; and
a processor configured to execute a signal feature correlation algorithm stored in a non-transitory computer-readable storage medium to perform operations comprising:
detecting a first feature of the first physiological signal, and
detecting a second feature of the second physiological signal based on an expected temporal correspondence between the first feature and the second physiological measurement.

13. The system of claim 12, wherein the first sensor includes one or more electrodes configured to collect an electrocardiogram (ECG) signal corresponding to an ECG measurement, and the second sensor includes an optical sensor configured to collect a photoplethysmogram (PPG) signal corresponding to a PPG measurement.

14. The system of claim 13, wherein the optical sensor includes:
a first light source configured to emit light at a red wavelength;
a second light source configured to emit light at an infrared wavelength; and
at least one photodetector configured to detect light reflected from tissue.

15. The system of claim 13, wherein the operations further comprise:
calculating a blood oxygen saturation value based at least on the second feature.

16. The system of claim 12, wherein the operations further comprise:
calibrating a timing offset between the first physiological signal and the second physiological signal based on a high signal-to-noise ratio segment of the second physiological signal.

17. The system of claim 12, wherein detecting the second feature comprises defining a search window based on a timing of the first feature and locating a local maximum or a local minimum within the search window.

18. A processing device, comprising:
one or more processors; and
a memory having stored computer-readable instructions that are executable by the one or more processors to perform operations comprising:
receiving a first physiological signal corresponding to a first physiological measurement and a second physiological signal corresponding to a second physiological measurement;
detecting a first feature of the first physiological signal; and
detecting a second feature of the second physiological signal based on an expected temporal correspondence between the first feature and the second physiological measurement.

19. The processing device of claim 18, wherein the first physiological signal is an electrocardiogram (ECG) waveform corresponding to an ECG measurement and the second physiological signal is a photoplethysmogram (PPG) waveform corresponding to a PPG measurement, and wherein the operations further comprise:
calculating a blood oxygen saturation value based at least on the second feature.

20. The processing device of claim 19, wherein detecting the second feature comprises defining a search window based on a timing of the first feature and a calibrated timing offset, and locating a local maximum or a local minimum within the search window.
